(19) United States
(12) Patent Application Publication
(10) Pub. No.: US 2026/0338895 A1
(43) Pub. Date: Oct. 29, 2026

(54) METHODS FOR ACCELERATED TIME-RESOLVED MULTI-ECHO MAGNETIC RESONANCE IMAGING BY AUGMENTING TEMPORAL CORRELATION

(71) Applicant: The General Hospital Corporation, Boston, MA (US)

(72) Inventors: Fuyixue Wang, Charlestown, MA (US); Zijing Dong, Charlestown, MA (US); Jonathan Polimeni, Cambridge, MA (US); Lawrence L. Wald, Cambridge, MA (US)

(21) Appl. No.: 19/120,193

(22) PCT Filed: Oct. 12, 2023
(86) PCT No.: PCT/US2023/076677

§ 371 Date: Apr. 10, 2025

Related U.S. Application Data
(60) Provisional application No. 63/415,575, filed on Oct. 12, 2022.

(51) Int. Cl. G01R 33/50; G01R 33/48; G01R 33/56; G01R 33/563

(52) U.S. Cl. CPC G01R 33/50; G01R 33/4826; G01R 33/5608; G01R 33/56341; G01R 33/56366

(57) ABSTRACT

Accelerated time-resolved multi-echo magnetic resonance imaging (MRI) uses augmenting of the temporal correlation across echoes, for example, by applying gradient blips that sample the spatiotemporal space in a new trajectory to reduce dead time and echo spacing. Additionally or alternatively, acceleration may be achieved by optimization of the encoding pattern to reduce temporal distance and/or using a spatiotemporal partial Fourier acquisition.

### CROSS-REFERENCE TO RELATED APPLICATIONS

This application claims the benefit of U.S. Provisional Patent Application Ser. No. 63/415,575, filed on Oct. 12, 2022, and entitled “METHODS FOR ACCELERATED TIME-RESOLVED MULTI-ECHO MAGNETIC RESONANCE IMAGING BY AUGMENTING TEMPORAL CORRELATION,” which is herein incorporated by reference in its entirety.

### STATEMENT OF FEDERALLY SPONSORED RESEARCH

This invention was made with government support under EB030006 awarded by the National Institutes of Health. The government has certain rights in the invention.

### BACKGROUND

Time-resolved multi-echo imaging is an emerging method in magnetic resonance imaging (MRI) that exploits the spatiotemporal correlation in the encoding process to enable fast multi-contrast multi-echo imaging. For example, echo planar time-resolved imaging (EPTI) is an acquisition method that addresses the long-standing limitations of the standard EPI acquisition, such as eliminating image distortion and blurring, while providing multi-contrast multi-echo images spaced at a small echo time (TE) increment of an echo spacing approximately 1 ms across the readout. In addition to Cartesian sampling, the time-resolved approach has also been used in non-Cartesian acquisitions. However, a large number of shots is required in EPTI acquisition to adequately sample the spatiotemporal encoding space, or k-t (i.e., k-TE) space, in order to accurately recover the multi-contrast multi-echo images. Reducing the number of shots will lead to increased dead time within the readout and, therefore, significantly lower temporal correlation between neighboring data, resulting in high noise amplification and image artifacts after image reconstruction. The need to acquire a large number of shots leads to low temporal resolution, high vulnerability to motion and physiological noise, and low SNR efficiency, limiting its use for many dynamic imaging applications, such as functional, diffusion, and perfusion MRI.

### SUMMARY OF THE DISCLOSURE

The present disclosure addresses the aforementioned drawbacks by providing a method for producing an image with a magnetic resonance imaging (MRI) system. The method includes acquiring multi-echo data with an MRI system using a pulse sequence that samples a hybrid space along a zigzag trajectory. The hybrid space includes a first axis along a temporal dimension and a second axis along a phase-encoding k-space dimension. A parameter of the pulse sequence is selected to augment temporal correlations across in echoes in the multi-echo data. One or more images may then be reconstructed from the multi-echo data.

The foregoing and other aspects and advantages of the present disclosure will appear from the following description. In the description, reference is made to the accompanying drawings that form a part hereof, and in which there is shown by way of illustration one or more embodiments. These embodiments do not necessarily represent the full scope of the invention, however, and reference is therefore made to the claims and herein for interpreting the scope of the invention.

### BRIEF DESCRIPTION OF THE DRAWINGS

FIGS. 1A and 1B illustrate an example echo spacing (ESP) reduction using a blip size minimized encoding, which can be used to reduce the temporal distance and augment temporal correlation in the spatiotemporal domain. The g-factor (noise amplification factor) of the spatiotemporal reconstruction is significantly reduced by reducing the ESP.

FIGS. 2A and 2B illustrate an example k-space sampling design that reduces the temporal distance by increasing the k_{y} distance of the neighboring data, which can augment temporal correlation and reduce g-factor and reconstruction errors.

FIGS. 3A and 3B show an example of using spatiotemporal partial Fourier (PF) acquisition to reduce the temporal distance and augment temporal correlation.

FIG. 4 is an example pulse sequence diagram for a gradient-echo and spin-echo (“GESE”) echo planar time-resolved imaging (“EPTI”) pulse sequence for one shot, which can be implemented with some embodiments described in the present disclosure.

FIG. 5 is a flowchart setting forth the steps of an example method for acquiring data using a pulse sequence that acquired data along a zigzag trajectory that is augmented to increase temporal correlations between echoes, and from which one or more images may be reconstructed.

FIGS. 6A-6D show k-t (TE) sampling schemes for (a) single-shot EPI, (b) multi-shot EPTI, (c) single-shot EPTI. EPTI exploits strong spatiotemporal correlation at sub-millisecond timescale to reconstruct full k-t data and provide multi-echo distortion-free images with distinct T₂* weightings. The proposed ss-EPTI encoding enables denser sampling and creates stronger data correlation, with complementary encoding to control aliasing, for higher temporal resolution, tSNR, CNR, and improved robustness to motion and physiological noise. (d) ss-EPTI reconstruction/process pipeline.

FIGS. 7A and 7B show (a) a comparison of tSNR efficiency (tSNR/VTR) of ss-EPI (TE=35 ms, TR=2.1 s, SMS 2), 3-shot EPTI (TE=4-62 ms, TR=1.8×3=5.4 s, SMS 3), and ss-EPTI (TE=4-62 ms, TE interval=0.49 ms, TR=2.6 s, SMS 2) at 2-mm isotropic resolution. ss-EPTI achieved ~30% higher tSNR efficiency over ss-EPI averaged across whole-brain and 70% higher than 3-shot EPTI, due to its efficient readout (less deadtime, ~2× more readout sampling) and robustness to motion and physiological noise. (b) ss-EPTI eliminates the geometric distortion artifacts common in EPI.

FIG. 8 illustrates that ss-EPTI achieves higher temporal stability with smaller temporal variations than EPI even when there was more subject motion during the scan. The temporal standard deviation (tSD) was normalized by the global mean intensity. The zoomed-in area (PE-dynamic view) shows that ss-EPTI eliminates the dynamic distortion/geometry changes common in EPI. These results suggest the increased temporal stability and motion robustness of ss-EPTI over EPI.

FIGS. 9A-9C show (a) Activation maps of EPI and ss-EPTI in a visual-task experiment (5.5-min runs at 3-mm: EPTI TR=1.7 s, EPI TR=1.5s). ss-EPTI shows higher t-score than EPI. (b) The average CNR in V1 mask across TEs. The optimal echo-combined EPTI image provides 50% higher CNR than EPI. The densely-sampled EPTI multi-echo images allow analysis of CNR change across TEs, peaked at TE=42 ms in V1 area (T2* of V1 area ~40-50 ms). A repeated ss-EPTI run shows consistent CNR improvement. (c) Effective signal dropout reduction using ss-EPTI and intra-voxel dephasing correction.

FIGS. 10A-10C shows examples of extracted signal components from ss-EPTI using ME-ICA. Left: spatial and temporal patterns. Middle: signals of different components (AS in black curve, ΔS/S in red) across echoes, which change differently depending on S0 (non BOLD) and R2* (BOLD) change. Right: goodness of fit using R2* or S0 (F (R2*), F (ΔSO)) and the S0 and R2* change (ΔR2*, ΔS0/S0). For BOLD components (a&b, strong ΔR2*), ΔS/S changes linearly with TE, while non-BOLD component (c, strong ΔS0) is more constant. The densely-sampled echoes also allow us to analyze the peak echo-time for BOLD components.

FIG. 11 is a block diagram of an example MRI system that can implement the methods described in the present disclosure.

### DETAILED DESCRIPTION

Described here are systems and methods for accelerated time-resolved multi-echo magnetic resonance imaging (MRI). In one aspect, acceleration is achieved by augmenting the temporal correlation across echoes, for example, by applying gradient blips that sample the spatiotemporal space in a new trajectory to reduce dead time and echo spacing. As another aspect, acceleration may be achieved by optimization of the encoding pattern to reduce temporal distance and/or using a spatiotemporal partial Fourier acquisition. In general, the disclosed systems and methods enable more efficient acquisition of the spatiotemporal encoding space, allowing for reliable reconstruction of the distortion/blurring-free multi-echo data acquired in significantly fewer number of shots, with as few as one shot for a single-shot acquisition. The more efficient acquisitions also enable improved SNR efficiency, robustness to motion and physiological noise, and higher temporal resolution for dynamic imaging applications such as functional MRI, perfusion MRI, and/or diffusion MRI. The disclosed systems and methods can also be used to improve the spatial resolution of time-resolved multi-echo MRI.

In some aspects, the systems and methods described in the present disclosure address the limitations of EPI and improve fMRI acquisition by developing a single-shot EPTI method. In this way, fast distortion-free imaging is provided with significantly improved temporal signal-to-noise ratio (tSNR), contrast-to-noise ratio (CNR), robustness to motion, reduced signal dropout, and multi-echo capability for denoising/analysis. The single-shot acquisition can be enabled by a k-t encoding that provides stronger correlation for improved reconstruction.

In general, the time-resolved imaging recovers undersampled data by exploiting the spatiotemporal correlation in a joint multi-echo or k-t (i.e., k-TE) space reconstruction. By augmenting the temporal correlation of the sampled data within the readout, the accuracy of the spatiotemporal reconstruction can be improved, which can enable accelerating time-resolved multi-echo MRI acquisitions. To augment the temporal correlation between neighboring data, a pulse sequence can be designed to sample time points (e.g., echoes) with reduced temporal distance.

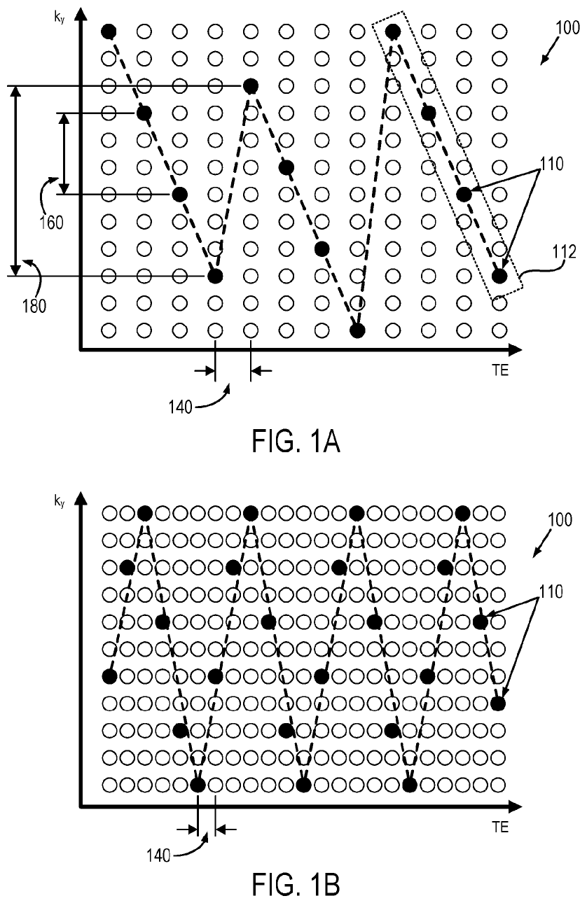

FIGS. 1A and 1B illustrate example sampling patterns 100 in k-t space. As one example, the k-t space is spanned by the k_{y} k-space dimension and the temporal dimension, t. In general, the sampling pattern 100 may take the form of a zigzag pattern. The sampling pattern 100 is created by selectively sampling data samples 110, which are phase encoding lines in k-space.

As noted and shown in FIG. 1A, the sampling pattern 100 used in EPTI may be a zigzag pattern that includes several linear section 112, or legs, that each include a plurality of temporally adjacent data samples 110. As shown in FIG. 1A, the data samples 110 on a given section 112 of the sampling pattern 100 are acquired in a temporally sequential manner and spaced apart in time by a first temporal spacing 140, and are spaced apart along the phase encoding dimension by a spacing 160. The spacing 160 in the phase encoding dimension is selected such that the data samples 110 on a given section 112 are interleaved in the phase encoding dimension with data samples 110 on a temporally adjacent section 112 of the sampling pattern 100. In other embodiments, two or more of the data samples 110 on a given section 112 of the sampling pattern 100 may be adjacent each other in the phase encoding dimension rather than spaced apart as shown in FIG. 1A.

The zigzag appearance to the sampling pattern 100 allows for neighboring data samples 110 to be acquired on the order of a millisecond apart such that they contain small B₀-inhomogeneity induced phase and T₂* decay. In the example illustrated in FIG. 1A, the first temporal spacing 140 is selected to be approximately 0.93 ms. As one example, the first temporal spacing 140 can be selected as the time interval of an echo spacing.

As one example, the temporal correlation of sampled data can be augmented by designing phase-encoding gradient blips that efficiently sample the k-t space in a new trajectory with reduced echo spacing. For instance, instead of repeatedly sampling multiple k_{y}-line sections that require large gradient blips (e.g., as illustrated by the gradient blip spacing 180 from one section of the sampling pattern 100 to the next) to jump through k-space, as illustrated in FIG. 1A, a k-space trajectory can be designed to continuously transverse through the k-t space up and down (e.g., along the k_{y}-direction) with smaller gradient blips, thereby reducing the dead time and achieving much shorter temporal spacing 140 (e.g., echo spacing), as illustrated in FIG. 1B. For instance, in the example illustrated in FIG. 1B the temporal spacing 140 may be 0.56 ms as compared to 0.93 ms in FIG. 1A. This approach significantly improves the sampling efficiency and increases the temporal correlation for improved reconstruction. For instance, the g-factor (noise amplification factor) of the spatiotemporal reconstruction using this blip-size minimized encoding is significantly reduced by reducing the echo spacing.

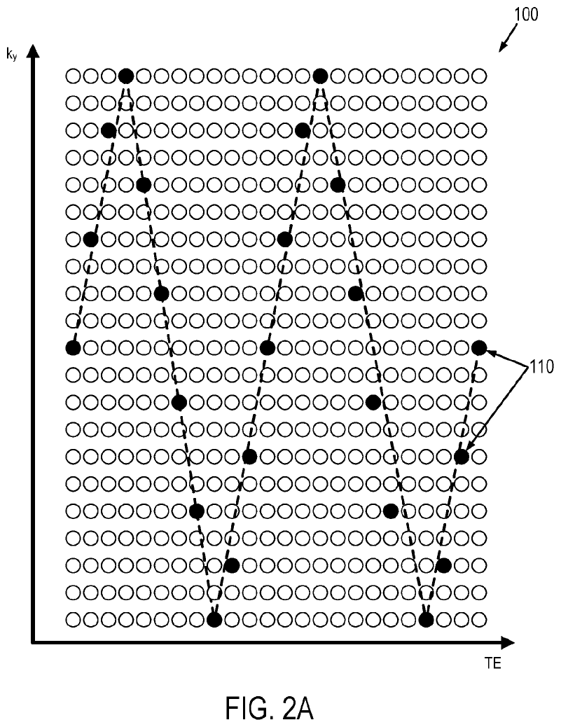

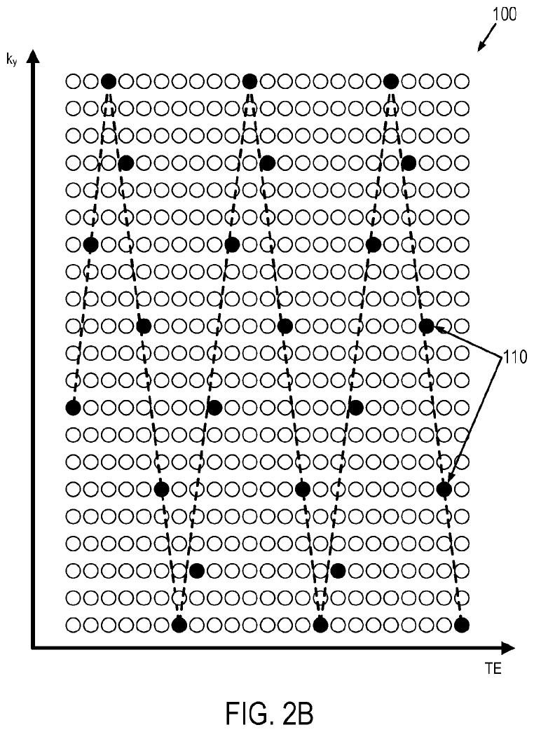

As another example, the temporal correlation of sampled data can be augmented by applying gradient blips to achieve an optimized spatiotemporal encoding pattern. For instance, an optimized spatiotemporal encoding pattern may include an encoding pattern that uses a larger k_{y} distance between neighboring data points (e.g., the spacing 160 as illustrated in FIG. 1A) to reduce the temporal distance (e.g., the temporal spacing 140 as illustrated in FIG. 1A). An example of such an optimized spatiotemporal encoding pattern is illustrated in FIGS. 2A and 2B. In the illustrated example, the temporal distance in k-t space is reduced by increasing the k_{y} distance of the neighboring data, such as by increasing the k_{y} distance from 4 (FIG. 2A) to 6 (FIG. 2B), which causes a reduction in the time distance from 6 (FIG. 2A) to 4 (FIG. 2B). This reduction in temporal distance can augment temporal correlation and reduce g-factor and reconstruction errors.

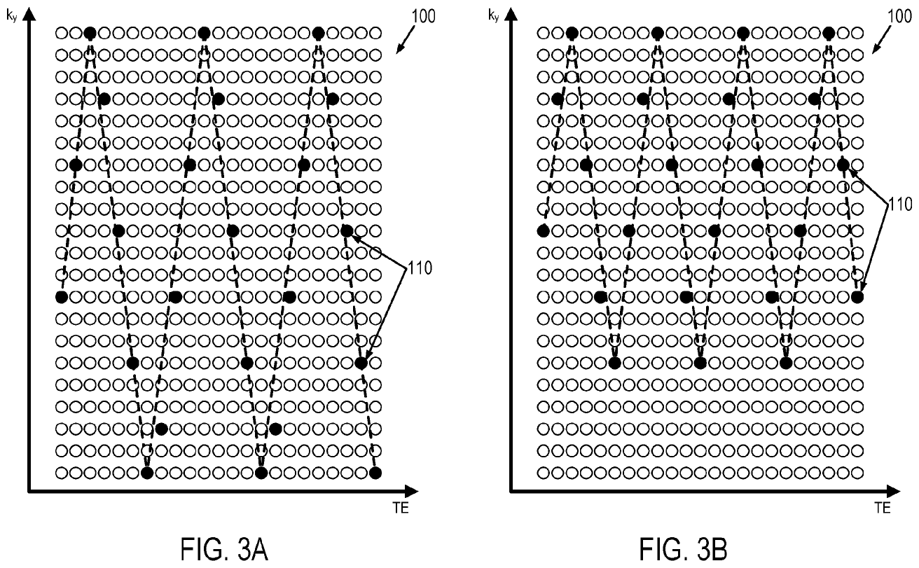

As yet another example, the temporal correlation of sampled data can be augmented by employing a k-t partial Fourier acquisition, as shown in FIGS. 3A and 3B. In the illustrated example, to reduce temporal distance (e.g., the temporal spacing 140 as illustrated in FIG. 1A or the temporal spacing between line sections), a segment of the k-t space is completely undersampled, as illustrated in FIG. 3B. The undersampled portion of k-t space can then be recovered using the conjugate symmetry property of k-space or any other partial Fourier reconstruction approaches, such as zero-filling or projection onto convex sets (POCS). Other undersampling schemes can also be used to augment the temporal correlation by undersampling a portion of k-t space, such as elliptic-centric filling. Non-Cartesian sampling strategies, such as spiral or radial sampling, can also be used to augment the temporal correction in the k-t space.

Using the systems and methods described in the present disclosure to generate higher temporal correlation in the sampled data, improved reconstruction performance can be achieved. This improved reconstruction performance can allow for reliable reconstruction of distortion/blurring-free multi-echo data acquired in significantly fewer number of shots, including in a single-shot acquisition.

In addition to reducing shot numbers, the improved spatiotemporal reconstruction provided by the disclosed systems and methods also means that larger k-t space coverage can be obtained in each shot. In this way, the disclosed systems and methods can additionally or alternatively be used to improve spatial resolution when using the same number of shots. The disclosed systems and methods can be used in both multi-shot and single-shot time-resolved imaging acquisitions, including 2D imaging and 3D volumetric imaging, as well as simultaneously multi-slice (SMS) imaging. For example, in 3D volumetric imaging, the disclosed systems and methods can be used in the hybrid kx-ky-kz-t space to augment the temporal correlation. In addition to Cartesian time-resolved imaging, the disclosed systems and methods can also be applied in non-Cartesian trajectories (e.g., spiral, radial, circular, PROPELLER).

The temporal correlation augmentation described in the present disclosure can be applied to all of the shots in an acquisition, or alternatively to only a subset of the acquisition shots. Additionally or alternatively, the temporal correlation augmentation can be applied to all of the spatiotemporal space, or alternatively to only part of the spatiotemporal space. Thus, in some instances, a variable density sampling pattern can be generated in the k-t space. A variable density sampling pattern can also improve reconstruction performance, especially when combining the reconstruction technique with a constrained reconstruction, such as compressed sensing, a low-rank approach, and/or machine learning-based reconstruction.

Advantageously, the discloses systems and methods enable magnetic resonance imaging techniques that provide distortion-free multi-echo images with improved SNR efficiency, robustness to motion and physiological noise, and higher temporal resolution for dynamic imaging applications, such as functional MRI (fMRI), perfusion, and/or diffusion MRI. As an example, the gain in reducing the number of shots (e.g., by achieving reliable single-shot time-resolved imaging, or otherwise) provides significant benefits, especially in applications where physiological noise are dominated and severe phase variations exist between different shots, which may include fMRI and diffusion MRI.

The disclosed systems and methods also improve the capability of time-resolved multi-echo MRI to achieve high spatial resolution imaging, since the high efficiency of the data acquisition techniques allows for a larger coverage of the k-t space in each shot. Densely-sampled multi-echo images, spanning across a wide range of TEs and spaced apart by a small TE increment, can be used to extract additional information content. For example, in fMRI, they can be used to further enhance the CNR through weighted combination of different echoes and also to remove physiological noise through extracting TE-dependent signal properties. Likewise, in diffusion MRI, the data acquisition techniques can provide diffusion-relaxometry measurements.

Accordingly, the disclosed systems and methods provide for accelerated time-resolved multi-echo MRI through augmenting the temporal correlation across echoes, for example, by applying gradient blips that sample the spatiotemporal space in a new trajectory to reduce dead time and echo spacing, by optimization of the encoding pattern to reduce temporal distance, and/or by using a spatiotemporal partial Fourier acquisition.

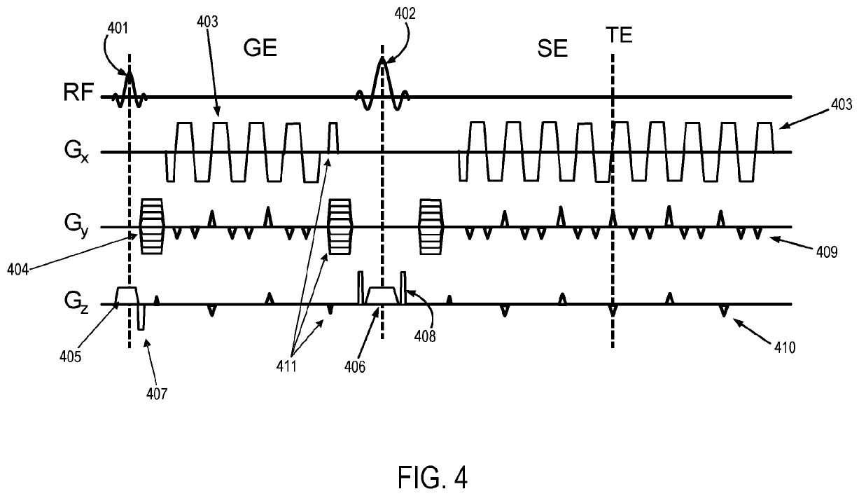

FIG. 4 shows an example of a pulse sequence that can be used to acquire data in accordance with some embodiments described in the present disclosure. The pulse sequence includes a radio frequency (RF) excitation pulse 401 that is played out in the presence of a slice-select gradient 405 in order to produce transverse magnetization in a prescribed imaging slice. The slice-select gradient 405 includes a rephasing lobe 407 that acts to rephase unwanted phase dispersions. A refocusing RF pulse 402 is later applied in the presence of another slice select gradient 406 in order to refocus transverse spin magnetization. In order to reduce unwanted stimulated echoes, crusher gradients 408 bridge the slice select gradient 406. Frequency encoding gradients 403 are applied in both a gradient echo (GE) readout occurring before the application of the refocusing RF pulse 402 and a spin echo (SE) readout occurring after the refocusing RF pulse 402. The target EPTI sampling pattern (e.g., the sampling patterns in FIG. 1B, 2A, 2B, 3A, and/or 3B) can be achieved by phase encoding gradients 404 and 409. Gradient blips 410 on the G, direction can be used to realize an SMS acquisition. The gradient module 411 can be used to compensate the unwanted phase dispersion due to encoding before refocusing.

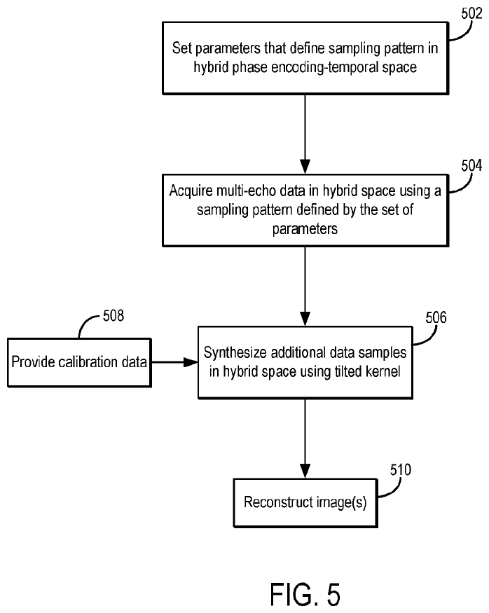

Referring now to FIG. 5, a flowchart is illustrated as setting forth the steps of an example method for reconstructing one or more images using an MRI system and an EPTI pulse sequence that acquires data using augmented temporal correlation to accelerate data acquisition as described in the present disclosure. The method includes setting parameters of the sampling pattern to be used for acquiring data, as indicated at step 502. The parameters can be selected via user input to a computer system that then communicates those parameters to the MRI system. For instance, the parameters can be entered via a user interface, which may be a graphical user interface. As described above, the parameters can include a temporal spacing between temporally adjacent data samples in each section of the hybrid space (e.g., k-t space) sampling pattern, a phase encoding spacing between temporally adjacent data samples in each section of the hybrid space sampling pattern, a second temporal spacing between temporally adjacent sections of the hybrid space sampling pattern, the number of sections in the hybrid space sampling pattern, the number of shots/segments of the phase encoding dimension, and so on.

As described above the parameters of the sampling pattern are optimized or otherwise selected to augment the temporal correlation of the sampling pattern, such that accelerated data acquisitions can be achieved. The temporal correlation may be augmented by selecting a gradient blip size that is optimized or otherwise selected to reduce dead time and thereby achieve much shorter temporal spacing (e.g., echo spacing), as shown in FIG. 1B. Additionally or alternatively, the temporal correlation may be augmented by selecting the phase-encoding spacing and/or temporal spacing (e.g., as shown in FIG. 2B) to optimize or otherwise select the spatiotemporal encoding for improved SNR efficiency, robustness to motion and physiological noise, shorter TR, larger coverage of the k-t space using the same number of shots, improved spatial resolution, combinations thereof, or the like. Other acceleration techniques can also be implemented, such as partial Fourier acquisitions in k-t space (e.g., as shown in FIG. 3B), SMS acquisitions, and so on.

After the parameters for the hybrid space (e.g., k-t space) sampling pattern have been selected, they are communicated to the MRI system, which is then operated to perform an EPTI pulse sequence to acquire data according to the defined hybrid space sampling pattern, as indicated at step 504. As described above, data can be acquired in one or more shots. The acquired data undersample the hybrid space in order to accelerate the data acquisition process; however, using the hybrid space sampling patterns described in the present disclosure B₀-inhomogeneity induced phase errors and T₂* decay related blurring are reduced.

Additional data in the hybrid space are then synthesized as indicated at step 506 in order to generate a data set that more fully samples the hybrid space. The additional data can be synthesized using a reconstruction kernel that spans the phase encoding and temporal dimensions in the hybrid space and that is tilted with respect to the phase encoding dimension, for example, as described in U.S. Pat. No. 11,022,665, which is herein incorporated by reference in its entirety. As one example, the reconstruction kernel can span a number of temporally adjacent sections of the sampling pattern such that the data samples included in the kernel provide at least one data sample for each phase encoding line spanned by the reconstruction kernel. For instance, the reconstruction kernel can span two adjacent sections of the sampling pattern when the interleaving in those two temporally adjacent sections fully sample the phase encoding dimension spanned by the kernel. Calibration data may be provided at step 508 to facilitate the synthesis of the additional data points. The calibration data can be low-resolution data acquired in a calibration scan.

One or more images of the subject are then reconstructed from the undersampled and additional data, as indicated at step 510. Any suitable reconstruction algorithm, such as those based on GRAPPA techniques and/or low-rank subspace methods, can be implemented for reconstruction. As one example, tilted-CAIPI reconstruction techniques described in U.S. Pat. No. 10,871,534, which is herein incorporated by reference in its entirety, can be used. As another example, a subspace-constrained reconstruction, such as those described in U.S. Pat. No. 11,022,665, can be implemented to reconstruct images.

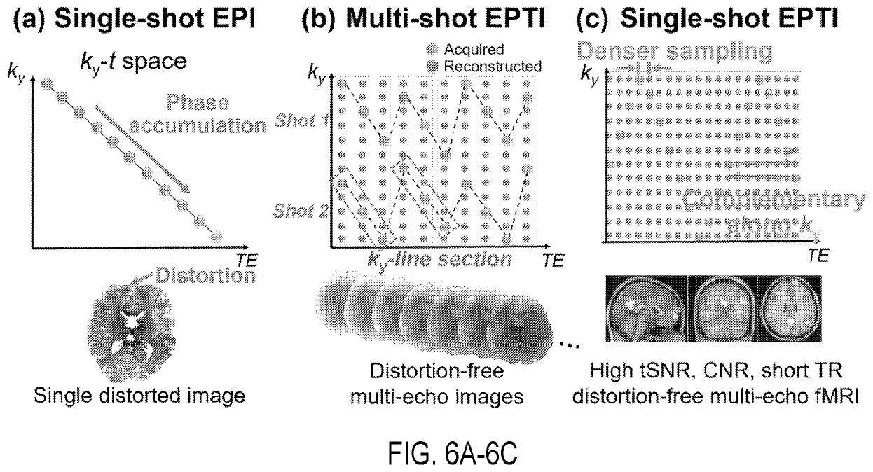

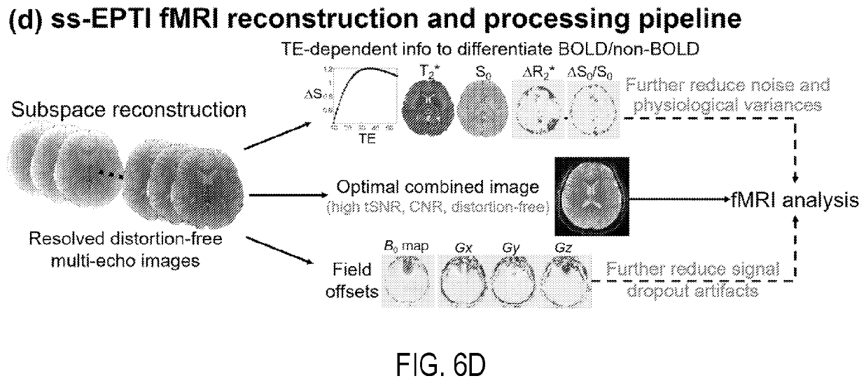

Conventional EPI generates a single distorted image after combining k-space samples evolving across time with accumulated B phase, as illustrated in FIG. 6A. EPTI uses spatiotemporal encodings (as illustrated in FIG. 6B) to exploit the data correlation and fully reconstruct the k-t (TE) space, generating >100 distortion-free multi-echo image across the EPTI readout. Original EPTI encoding acquires different k_{y}-line-sections to ensure regular sampling to apply GRAPPA kernel, which requires large gradient blips, especially for single-shot acquisitions, leading to deadtime and compromised data correlation. To achieve a single-shot EPTI acquisition, a zigzag trajectory that continuously traverses through the k-t (TE) space with minimal gradient blips is used, as illustrated in FIG. 6C. In this way, the deadtime is reduced to achieve much shorter echo spacing with stronger spatiotemporal correlation for improved reconstruction (e.g., ~40% denser sampling, echo-spacing 0.93 ms reduced to 0.55 ms at 2-mm). The samplings can be complementary along k_{y} to better use the multi-channel coil information. SMS imaging can also be integrated to increase temporal resolution.

An example ss-EPTI reconstruction and processing pipeline for fMRI is shown in FIG. 6D. First, a multi-echo subspace reconstruction is employed to exploit the correlation in the k-t (TE) space and resolve multi-echo images completely free from static and dynamic distortions. The multi-echo images are then combined based on the T₂* of every voxel to generate CNR-optimal images. Intra-voxel dephasing correction can also be incorporated into the reconstruction pipeline to reduce the dropout artifacts. Additionally, the multi-echo images can be used to extract TE-dependency information to identify and remove non-BOLD variations for motion and physiological-noise/artifacts reduction using multi-echo independent component analysis (ME-ICA).

In an example study, visual-task and resting-state fMRI experiments were performed on healthy volunteers using ss-EPTI and standard ss-EPI. Both tSNR and CNR were evaluated in this example without denoising/smoothing.

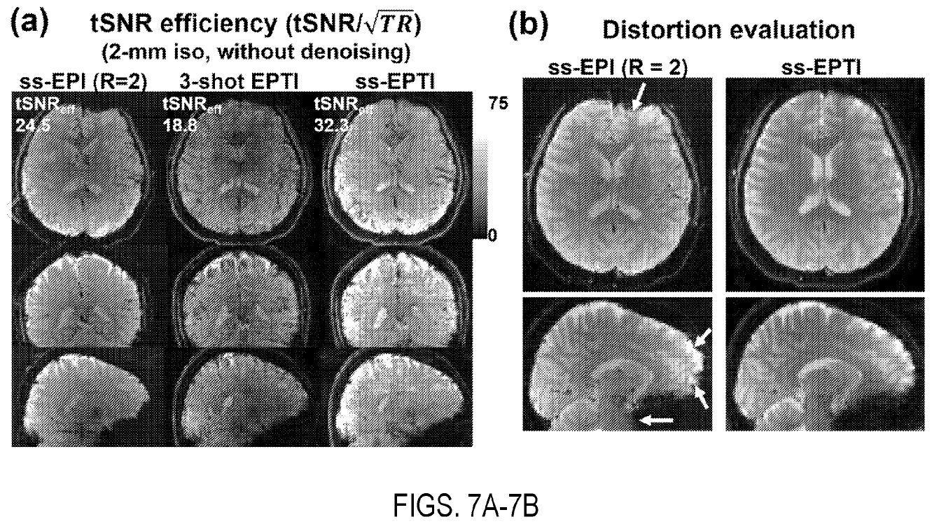

FIG. 7A shows the tSNR efficiency (tSNR/VTR) of conventional ss-EPI (TR minimized), 3-shot EPTI, and ss-EPTI. ss-EPTI provides 30% higher tSNR efficiency over ss-EPI and 70% higher than 3-shot EPTI, due to its high sampling efficiency (~2× more sampling time than ss-EPI/TR with only ~20% increase in TR) and increased robustness to motion and physiological noise. FIG. 7B shows that ss-EPTI eliminates geometric distortion common in ss-EPI even with short effective echo-spacing.

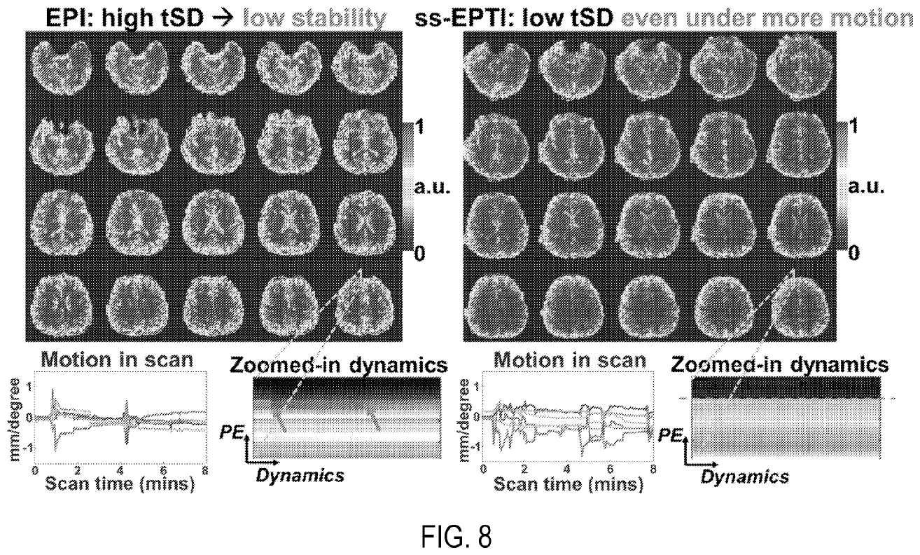

The elimination of motion-related dynamic distortion of ss-EPTI provides higher temporal stability (FIG. 8) even with more subject motion. The example zoomed-in area shows the local variation of the brain contour in ss-EPI and static image geometry of ss-EPTI.

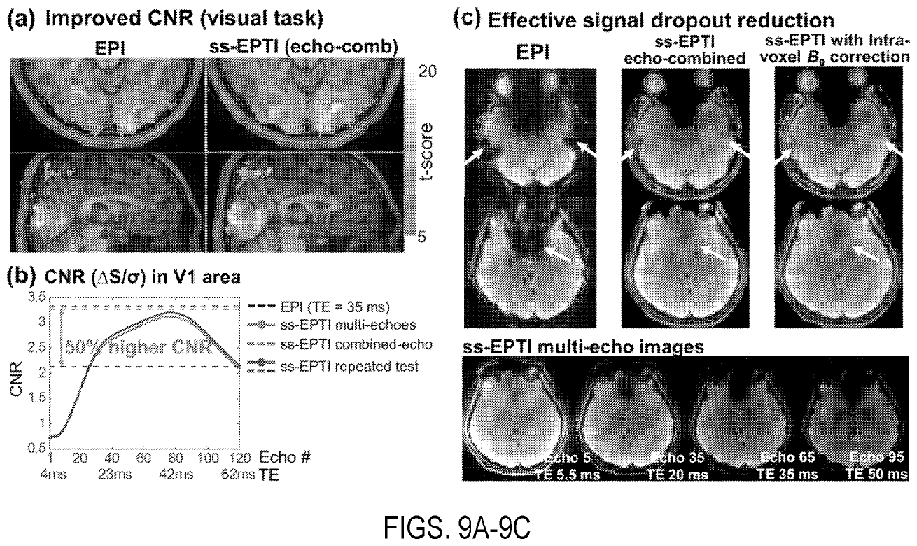

In visual-task experiment, ss-EPTI shows improved CNR than EPI with higher t-score (FIG. 9A). The optimal echo-combined EPTI images provide 50% higher CNR in V1 area. The dense EPTI multi-echo images enable investigation of CNR across TEs (FIG. 9B), peaked at TE=42 ms in V1 area. A repeated EPTI run was tested, and consistent results were found. FIG. 9C presents the effectively reduced signal dropout using ss-EPTI, benefiting from the acquired early-TE images and intra-voxel dephasing correction.

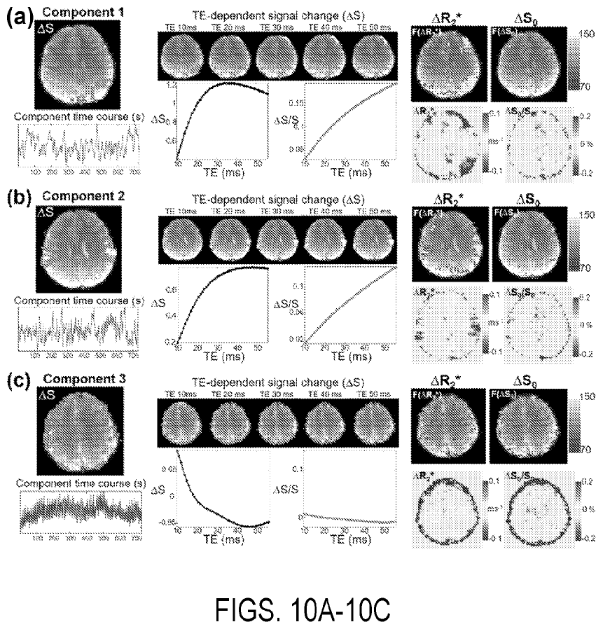

ss-EPTI multi-echo images can be used to investigate TE-dependency and differentiate BOLD/non-BOLD components for noise removal. The signal (ΔS shown in black curve or ΔS/S in red) change differently across echoes depending on the S0 (non-BOLD) and R₂* (BOLD) change. For BOLD components (FIGS. 10A and 10B, strong R₂* change), percent signal change (ΔS/S) changes linearly with TE, while the non-BOLD component (FIG. 10C, strong So change) is more constant across TEs. The densely-sampled multi-echo images also allow analysis of peak echo-time for different BOLD components.

Single-shot EPTI improves the fMRI acquisition by providing high-quality distortion-free images, with higher tSNR, CNR efficiency, temporal stability, reduced signal dropout, and multi-echo capability for denoising.

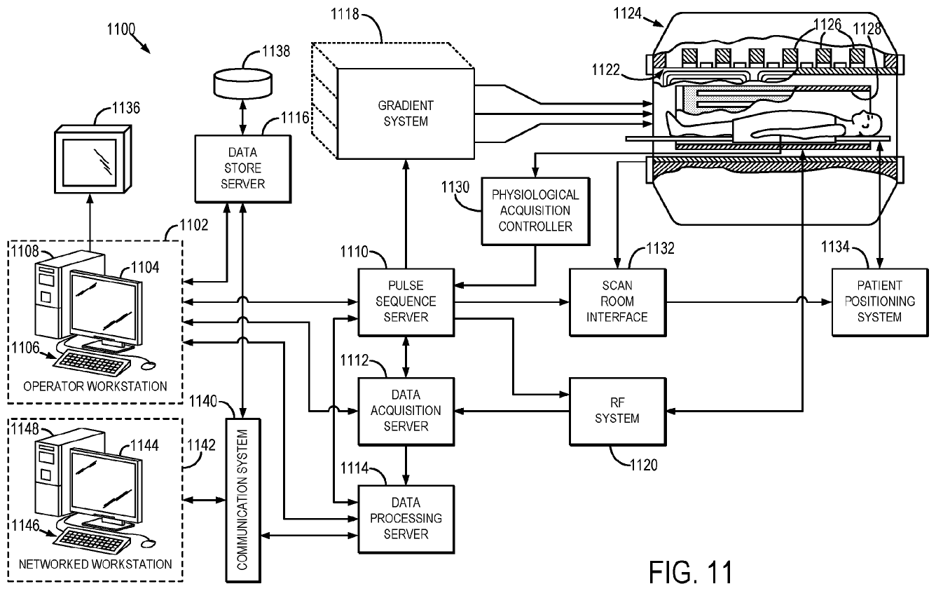

Referring particularly now to FIG. 11, an example of an MRI system 1100 that can implement the methods described here is illustrated. The MRI system 1100 includes an operator workstation 1102 that may include a display 1104, one or more input devices 1106 (e.g., a keyboard, a mouse), and a processor 1108. The processor 1108 may include a commercially available programmable machine running a commercially available operating system. The operator workstation 1102 provides an operator interface that facilitates entering scan parameters into the MRI system 1100. The operator workstation 1102 may be coupled to different servers, including, for example, a pulse sequence server 1110, a data acquisition server 1112, a data processing server 1114, and a data store server 1116. The operator workstation 1102 and the servers 1110, 1112, 1114, and 1116 may be connected via a communication system 1140, which may include wired or wireless network connections.

The pulse sequence server 1110 functions in response to instructions provided by the operator workstation 1102 to operate a gradient system 1118 and a radiofrequency (“RF”) system 1120. Gradient waveforms for performing a prescribed scan are produced and applied to the gradient system 1118, which then excites gradient coils in an assembly 1122 to produce the magnetic field gradients Gₓ, G_{y}, and G_{z} that are used for spatially encoding magnetic resonance signals. The gradient coil assembly 1122 forms part of a magnet assembly 1124 that includes a polarizing magnet 1126 and a whole-body RF coil 1128.

RF waveforms are applied by the RF system 1120 to the RF coil 1128, or a separate local coil to perform the prescribed magnetic resonance pulse sequence. Responsive magnetic resonance signals detected by the RF coil 1128, or a separate local coil, are received by the RF system 1120. The responsive magnetic resonance signals may be amplified, demodulated, filtered, and digitized under direction of commands produced by the pulse sequence server 1110. The RF system 1120 includes an RF transmitter for producing a wide variety of RF pulses used in MRI pulse sequences. The RF transmitter is responsive to the prescribed scan and direction from the pulse sequence server 1110 to produce RF pulses of the desired frequency, phase, and pulse amplitude waveform. The generated RF pulses may be applied to the whole-body RF coil 1128 or to one or more local coils or coil arrays.

The RF system 1120 also includes one or more RF receiver channels. An RF receiver channel includes an RF preamplifier that amplifies the magnetic resonance signal received by the coil 1128 to which it is connected, and a detector that detects and digitizes the I and Q quadrature components of the received magnetic resonance signal. The magnitude of the received magnetic resonance signal may, therefore, be determined at a sampled point by the square root of the sum of the squares of the I and Q components:

M=\sqrt{I²+Q²};
and the phase of the received magnetic resonance signal may also be determined according to the following relationship:

$$φ = {tan}^{- 1} ( \frac{Q}{I} ) .$$

The pulse sequence server 1110 may receive patient data from a physiological acquisition controller 1130. By way of example, the physiological acquisition controller 1130 may receive signals from a number of different sensors connected to the patient, including electrocardiogramh (“ECG”) signals from electrodes, or respiratory signals from a respiratory bellows or other respiratory monitoring devices. These signals may be used by the pulse sequence server 1110 to synchronize, or “gate,” the performance of the scan with the subject's heart beat or respiration.

The pulse sequence server 1110 may also connect to a scan room interface circuit 1132 that receives signals from various sensors associated with the condition of the patient and the magnet system. Through the scan room interface circuit 1132, a patient positioning system 1134 can receive commands to move the patient to desired positions during the scan.

The digitized magnetic resonance signal samples produced by the RF system 1120 are received by the data acquisition server 1112. The data acquisition server 1112 operates in response to instructions downloaded from the operator workstation 1102 to receive the real-time magnetic resonance data and provide buffer storage, so that data is not lost by data overrun. In some scans, the data acquisition server 1112 passes the acquired magnetic resonance data to the data processor server 1114. In scans that require information derived from acquired magnetic resonance data to control the further performance of the scan, the data acquisition server 1112 may be programmed to produce such information and convey it to the pulse sequence server 1110. For example, during pre-scans, magnetic resonance data may be acquired and used to calibrate the pulse sequence performed by the pulse sequence server 1110. As another example, navigator signals may be acquired and used to adjust the operating parameters of the RF system 1120 or the gradient system 1118, or to control the view order in which k-space is sampled. In still another example, the data acquisition server 1112 may also process magnetic resonance signals used to detect the arrival of a contrast agent in a magnetic resonance angiography (“MRA”) scan. For example, the data acquisition server 1112 may acquire magnetic resonance data and processes it in real-time to produce information that is used to control the scan.

The data processing server 1114 receives magnetic resonance data from the data acquisition server 1112 and processes the magnetic resonance data in accordance with instructions provided by the operator workstation 1102. Such processing may include, for example, reconstructing two-dimensional or three-dimensional images by performing a Fourier transformation of raw k-space data, performing other image reconstruction algorithms (e.g., iterative or backprojection reconstruction algorithms), applying filters to raw k-space data or to reconstructed images, generating functional magnetic resonance images, or calculating motion or flow images.

Images reconstructed by the data processing server 1114 are conveyed back to the operator workstation 1102 for storage. Real-time images may be stored in a data base memory cache, from which they may be output to operator display 1102 or a display 1136. Batch mode images or selected real time images may be stored in a host database on disc storage 1138. When such images have been reconstructed and transferred to storage, the data processing server 1114 may notify the data store server 1116 on the operator workstation 1102. The operator workstation 1102 may be used by an operator to archive the images, produce films, or send the images via a network to other facilities.

The MRI system 1100 may also include one or more networked workstations 1142. For example, a networked workstation 1142 may include a display 1144, one or more input devices 1146 (e.g., a keyboard, a mouse), and a processor 1148. The networked workstation 1142 may be located within the same facility as the operator workstation 1102, or in a different facility, such as a different healthcare institution or clinic.

The networked workstation 1142 may gain remote access to the data processing server 1114 or data store server 1116 via the communication system 1140. Accordingly, multiple networked workstations 1142 may have access to the data processing server 1114 and the data store server 1116. In this manner, magnetic resonance data, reconstructed images, or other data may be exchanged between the data processing server 1114 or the data store server 1116 and the networked workstations 1142, such that the data or images may be remotely processed by a networked workstation 1142.

The present disclosure has described one or more preferred embodiments, and it should be appreciated that many equivalents, alternatives, variations, and modifications, aside from those expressly stated, are possible and within the scope of the invention.

1. A method for producing an image with a magnetic resonance imaging (MRI) system, the steps comprising:
a) acquiring multi-echo data with an MRI system using a pulse sequence that samples a hybrid space along a sampling trajectory, wherein the hybrid space comprises a first axis along a temporal dimension and a second axis along a phase-encoding k-space dimension, wherein at least one parameter of the pulse sequence is selected to augment temporal correlations across in echoes in the multi-echo data; and
b) reconstructing at least one image from the multi-echo data.

2. The method of claim 1, wherein the at least one parameter of the pulse sequence comprises a gradient blip magnitude.

3. The method of claim 2, wherein the gradient blip magnitude is selected to reduce dead time and echo spacing in the sampling trajectory.

4. The method of claim 2, wherein the at least one parameter of the pulse sequence further comprises a number of gradient blips used for phase encoding.

5. The method of claim 4, wherein the number of gradient blips and the gradient blip magnitude are selected to continuously traverse the hybrid space with the sampling trajectory to reduce dead time and echo spacing.

6. The method of claim 1, wherein the parameter of the pulse sequence is at least one of a phase-encoding sample spacing or a temporal sample spacing that define an encoding pattern of the sampling trajectory.

7. The method of claim 6, wherein the temporal sample spacing is selected to reduce temporal distance in the encoding pattern of the sampling trajectory.

8. The method of claim 7, wherein the phase-encoding sample spacing is increased to cause the temporal distance to be reduced.

9. The method of claim 1 wherein the acquired multi-echo data undersample the hybrid space and step b) includes synthesizing additional data in the hybrid space using a reconstruction kernel that spans the phase-encoding k-space dimension and the temporal dimension, wherein the image is reconstructed from the acquired multi-echo data and the additional data.

10. The method of claim 9 wherein the reconstruction kernel is a tilted reconstruction kernel that is oriented at an angle with respect to the phase-encoding k-space dimension in the hybrid space.

11. The method of claim 1, wherein the at least one image is reconstructed from the multi-echo data using a low-rank subspace approach that reconstructs multi-echo images from the multi-echo data.

12. The method of claim 1 wherein the sampling trajectory comprises a zigzag trajectory with a plurality of temporally adjacent linear sections each comprising a plurality of temporally adjacent data samples that are spaced apart along the phase-encoding k-space dimension by a phase encoding spacing and are spaced apart along the temporal dimension by a temporal spacing.

13. The method of claim 12 wherein the temporal spacing is less than a few milliseconds.

14. The method of claim 12 wherein the phase encoding spacing is selected such that data samples in the plurality of temporally adjacent linear sections are interleaved in the phase encoding dimension.

15. The method of claim 1, wherein the multi-echo data are acquired by sampling the hybrid space with the sampling trajectory using a spatiotemporal partial Fourier acquisition to augment the temporal correlations.

16. The method of claim 1, wherein the multi-echo data are acquired using a single shot acquisition of the pulse sequence.

17. The method of claim 1, wherein the multi-echo data are acquired using a plurality of shots of the pulse sequence.

18. The method of claim 1, wherein sample points on the sampling trajectory are complementary along the phase-encoding k-space dimension.

19. The method of claim 1, wherein the multi-echo data comprise time-resolved multi-echo data.

20. The method of claim 19, wherein the pulse sequence comprises an echo planar time-resolved imaging (EPTI) pulse sequence.

21. The method of claim 19, wherein the pulse sequence comprises a three-dimensional multi-echo imaging pulse sequence.

22. The method of claim 19, wherein the pulse sequence comprises a multi-echo pulse sequence with a simultaneous multislice acquisition.

23. The method of claim 19, wherein the sampling trajectory comprises a non-Cartesian spiral sampling trajectory.

24. The method of any one of claim 1-11 or 13-22, wherein the sampling trajectory is a zigzag trajectory.
